(19) United States
(12) Patent Application Publication
(10) Pub. No.: US 2022/0301671 A1
(43) Pub. Date: Sep. 22, 2022

(54) COGNITIVE ENGINE COMPILATION OF ELECTRONIC MEDICAL RECORDS BASED ON PATIENT CONDITION

(71) Applicant: Nimble Co., LLC d/b/a PURVIEW, Annapolis, MD (US)

(72) Inventors: Leslie S. TRACHTMAN, Crownsville, MD (US); Fauze Valério POLPETA, Florianopolis (BR)

(73) Assignee: Nimble Co., LLC d/b/a PURVIEW, Annapolis, MD (US)

(21) Appl. No.: 17/655,263

(22) Filed: Mar. 17, 2022

Related U.S. Application Data
(60) Provisional application No. 63/163,123, filed on Mar. 19, 2021.

(51) Int. Cl. G16H 15/00; G16H 10/60; G16H 50/20

(52) U.S. Cl. CPC G16H 15/00; G16H 10/60; G16H 50/20

(57) ABSTRACT

Medical care is facilitated by providing a cognitive engine to compile electronic medical data records of a patient related to a medical condition into an electronic case abstraction. A processor(s) scans a set of electronic files for electronic medical data records of an individual related to a medical condition, where the scanning identifies electronic records which include different file types with different unique identifiers, and which are from different electronic medical records systems. The processor(s) further electronically compiles the identified electronic medical data records of the individual related to the medical condition into the electronic case abstraction, including associating a common case identifier with the identified electronic medical data records of the individual related to the medical condition. Further, the processor(s) electronically provides for display on a display screen in a consolidated view, the electronic case abstraction to facilitate medical care of the individual.

### CROSS-REFERENCE TO RELATED APPLICATION

This application claims the benefit of U.S. provisional patent application Ser. No. 63/163,123, filed Mar. 19, 2021, entitled “Web Based Remote Universal Medical Records Uploader Organizer and File Router”, which is hereby incorporated herein by reference in its entirety.

### BACKGROUND

Today, healthcare organizations typically store character-based healthcare records, such as clinical records, in an electronic health record (EHR) system. EHR systems are provided by a variety of vendors, which typically store medical records in their systems in a proprietary format. This inhibits EHR systems from different vendors from being able to communicate to transfer records between them.

Further, EHR systems typically do not natively enable the storage of medical images. Medical images are often obtained in a Digital Imaging and Communications in Medicine (DICOM) format, which is the standard for communication and management of medical imaging information and related data. DICOM medical imaging information is often stored in a Picture Archiving and Communication System (PACS), such as available from multiple providers. DICOM files are generally much larger than clinical text files, and sometimes as large as several gigabytes in volume.

Further, EHR systems do not natively enable the storage of digital pathology files. Unlike medical image scans, there currently is no standard format for a pathology file. Pathology files can exist in a multitude of proprietary formats, and are generally stored in their native file format, or in certain cases, in a purpose-built proprietary pathology storage system. Pathology files can be even larger than DICOM files, often reaching 5-10 gigabytes in volume.

### SUMMARY

Certain shortcomings of the prior art are overcome and additional advantages are provided through the provision, in one or more aspects, of a computer-implemented method for facilitating medical care, which includes scanning, by one or more processors, a set of electronic files for electronic medical data records of an individual related to a medical condition. The scanning identifies electronic medical data records of the individual related to the medical condition which include different file types with different unique identifiers, and which are from different electronic medical records systems. The method further includes electronically compiling, by the one or more processors, the identified electronic medical data records of the individual related to the medical condition into an electronic case abstraction, including associating a common case identifier with the identified electronic medical data records of the individual related to the medical condition. Further, the method includes electronically providing, by the one or more processors, for display on a screen in a consolidated view, the electronic case abstraction to facilitate medical care of the individual.

In one or more embodiments, the identified electronic medical data records of the individual related to the medical condition compiled for the electronic case abstraction of different file types include two or more of an electronic pathology-type file, an electronic radiology-type file, and an electronic clinical-type file.

In one or more embodiments, the scanning includes electronically scanning the set of electronic files using a heuristic algorithm and a database of known medical record patterns, to identify the electronic medical data records of the individual related to the medical condition which include different file types with different unique identifiers, and which are from different electronic medical records systems.

In one or more embodiments, the computer-implemented method further includes obtaining, by the one or more processors, the set of electronic files from the different electronic medical records systems, wherein two or more electronic medical records systems of the different electronic medical records systems are incompatible electronic medical records systems.

In one or more embodiments, the method further includes categorizing by file type and file context the identified electronic medical data records of the individual related to the medical condition, wherein categorizing by file context results in associating two or more identified electronic medical data records of different file type together in the electronic case abstraction. In one embodiment, the categorizing includes comparing electronic files of the set of electronic files to known file structures, including known pathology record structures, known radiology record structures, and known clinical record structures to facilitate categorizing the identified electronic medical data records by file type. In one or more implementations, the computer-implemented method further includes rules-based forwarding of the identified electronic medical data records to appropriate data storage in accordance with respective categorized file types of the identified electronic medical data records.

In one or more embodiments, the electronically compiling, by the one or more processors, the identified electronic medical data records of the individual related to the medical condition into the electronic case abstraction includes electronically compiling, by the one or more processors, the electronic case abstraction by extracting metadata from the identified electronic medical data records of the individual related to the medical condition, and processing the metadata to facilitate the electronically compiling of the electronic case abstraction. In one or more embodiments, extracting the metadata includes parsing structured metadata associated with one or more electronic medical data records of the identified electronic medical data records. In one or more embodiments, the computer-implemented method further includes using a natural language processor to extract unstructured metadata from one or more other electronic medical data records of the identified medical data records to obtained parsed metadata for the other electronic medical data record(s) for use in electronically compiling the electronic case abstraction.

In one or more embodiments, the computer-implemented method further includes training one or more machine learning models to make one or more recommendations to facilitate medical care of individuals based on electronic case abstractions, and electronically providing, by the one or more processors, using the machine learning model(s), a recommendation to facilitate medical care of the individual related to the medical condition. In one embodiment, the electronically providing includes electronically providing, by the one or more processors, using the machine learning model(s), a medical referral for the individual related to the medical condition to a specialist.

Computer systems and computer program products relating to one or more aspects are also described and claimed herein. Further, services relating to one or more aspects are also described and may be claimed herein.

Additional features and advantages are realized through the techniques described herein. Other embodiments and aspects of the invention are described in detail herein and are considered a part of the claimed aspects.

### BRIEF DESCRIPTION OF THE DRAWINGS

One or more aspects of the present invention are particularly pointed out and distinctly claimed as examples in the claims at the conclusion of the specification. The foregoing and other objects, features, and advantages of the invention are apparent from the following detailed description taken in conjunction with the accompanying drawings in which:

FIG. 1 depicts one example of a computing environment to incorporate and use one or more aspects of the present invention;

FIG. 2 depicts one embodiment of a workflow illustrating certain aspects of one or more embodiments of the present invention;

FIG. 3A depicts one embodiment of a collector workflow illustrating certain aspects of one or more embodiments of the present invention;

FIG. 3B depicts another embodiment of a collector workflow illustrating certain aspects of one or more embodiments of the present invention;

FIG. 4 depicts one embodiment of a compiler workflow illustrating certain aspects of one or more embodiments of the present invention;

FIG. 5 depicts one embodiment of a presenter workflow illustrating certain aspects of one or more embodiments of the present invention;

FIG. 6, illustrates one embodiment of routing destinations for identified electronic medical data records of an electronic case abstract, in accordance with one or more aspects of the present invention;

FIG. 7 depicts another example of a computing environment to incorporate and use one or more aspects of the present invention;

FIG. 8A depicts another embodiment of workflow illustrating certain aspects of one or more embodiments of the present invention;

FIG. 8B depicts further details of one embodiment of a computing environment and workflow illustrating certain aspects of one or more embodiments of the present invention;

FIG. 9 illustrates another example of a computing environment to incorporate and use one or more aspects of the present invention; and

FIG. 10 depicts another example of a computing environment to incorporate and use one or more aspects of the present invention.

### DETAILED DESCRIPTION

The accompanying figures, which are incorporated in and form a part of this specification, further illustrate the present invention and, together with this detailed description of the invention, serve to explain aspects of the present invention. Note in this regard that descriptions of well-known systems, devices, processing techniques, etc., are omitted so as to not unnecessarily obscure the invention in detail. It should be understood, however, that the detailed description and this specific example(s), while indicating aspects of the invention, are given by way of illustration only, and not limitation. Various substitutions, modifications, additions, and/or other arrangements, within the spirit or scope of the underlying inventive concepts will be apparent to those skilled in the art from this disclosure. Note further that numerous inventive aspects or features are disclosed herein, and unless inconsistent, each disclosed aspect or feature is combinable with any other disclosed aspect or feature as desired for a particular application of the concepts disclosed herein.

Note also that illustrative embodiments are described below using specific code, designs, architectures, protocols, layouts, schematics, or tools only as examples, and not by way of limitation. Furthermore, the illustrative embodiments are described in certain instances using particular software, tools, or data processing environments only as example for clarity of description. The illustrative embodiments can be used in conjunction with other comparable or similarly purposed structures, systems, applications, or architectures. One or more aspects of an illustrative embodiment can be implemented in software, hardware, or a combination thereof.

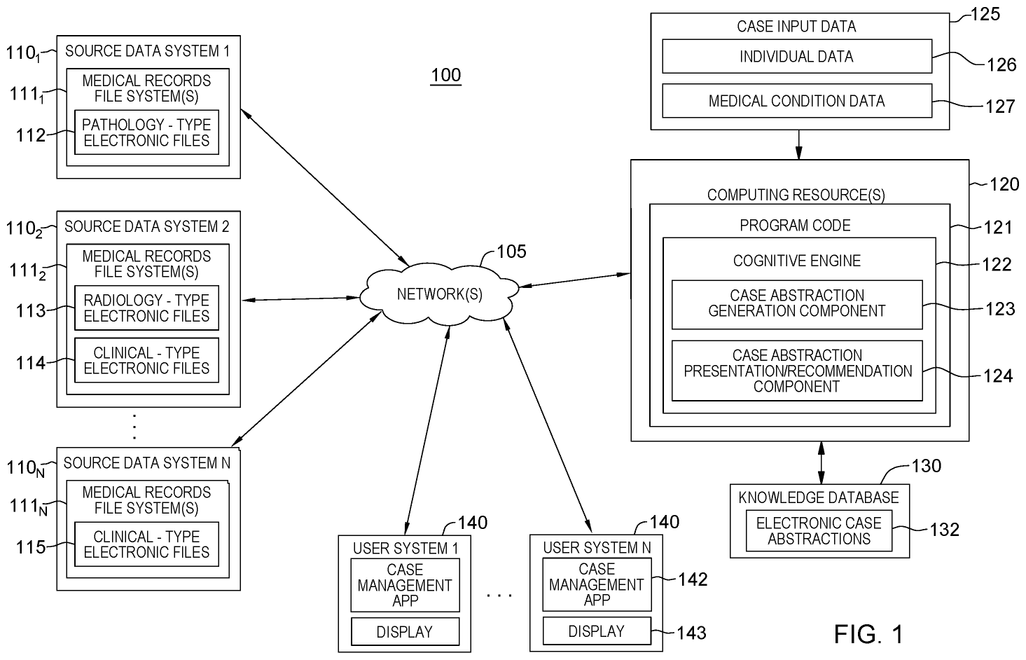

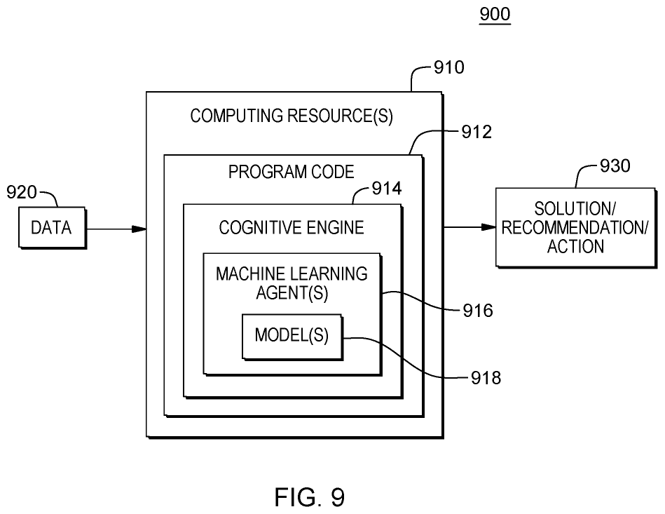

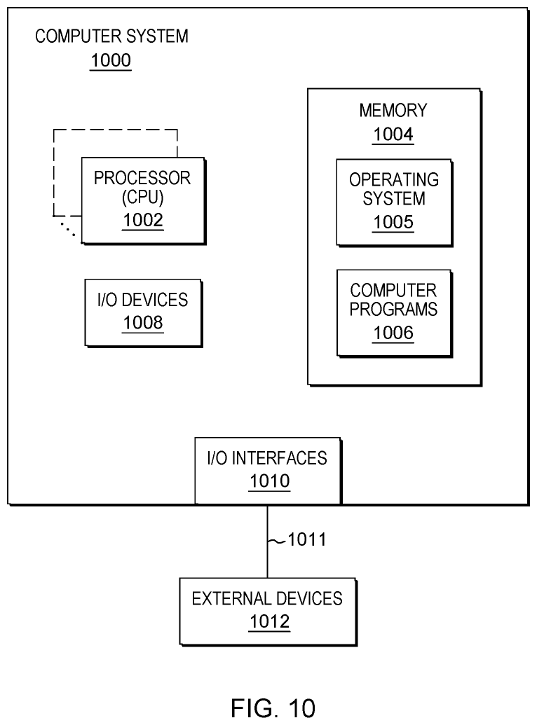

As understood by one skilled in the art, program code, as referred to in this application, can include both software and hardware. For example, program code in certain embodiments of the present invention can utilize a software-based implementation of certain functions described, while other embodiments can include (at least in part) fixed function hardware. Certain embodiments can combine both types of program code. Examples of program code, also referred to as one or more programs, are depicted in FIG. 1 as program code 121, in FIG. 9 as program code 912, and in FIG. 10 as computer programs 1006, which are illustrated by way of example only.

As noted, electronic medical records often reside in heterogeneous formats in a multitude of different, and incompatible, electronic medical records systems. As a result, when a patient's records are required by a third party, for instance, for diagnosis and/or treatment, it is often a time-consuming process to physically gather and transfer the records, and once received, the records are typically not displayable in a single consolidated view. Upon transfer, there also remains the requirement that the medical records be properly securely stored at the receiving facility.

Due to the incompatibility of different EHR systems and differences between DICOM and pathology files, it is often problematic to transfer medical records from a first location to a second location, without either printing and mailing or faxing text files, and mailing CDs or DVDs with DICOM or pathology files. Once received, these files then need to be re-digitized or loaded, and attributed to the correct patient, before being securely stored. This transfer of digital information to paper, fax, CD, DVD, etc., and mailing, can introduce errors, time delays, and often reduces quality of the medical records transferred. Further, once transferred, the different electronic file types and formats are not able to be presented in a single system view, often requiring, for instance, a treating physician to access a set of heterogeneous applications in order to review a complete patient record history.

Disclosed herein are computer-implemented methods, computer systems and computer program products for facilitating patient medical care by remotely obtaining, compiling and presenting electronic medical data records of any type and format, from different electronic medical records systems, in a single case-dependent electronic abstraction. Embodiments of the present invention include program code executing on one or more processors which electronically scans a set of electronic files for electronic medical data records of an individual (e.g., a patient) related to a specified medical condition. The scanning identifies electronic medical data records of the individual related to the medical condition which include different file types with different unique identifiers, and which are from different electronic medical records systems. Further, program code executing on the one or more processors electronically compiles the identified electronic medical data records of the individual related to the medical condition into an electronic case abstraction, including associating a common case identifier with the identified medical data records of the individual related to the medical condition. In addition, program code executing on the one or more processors electronically provides for display on a display screen, in a consolidated view, the electronic case abstraction to facilitate medical care of the individual. The program code includes embedded electronic file downloaders, viewers and routers, which allow each file type of the identified electronic medical data records to be displayed on the display screen, for example, by user selection thereof, through the electronic case abstraction.

By way of example, FIG. 1 depicts one embodiment of a computing environment, generally denoted 100, to incorporate and use one or more aspects of the present invention. In one or more embodiments, computing environment 100 includes one or more source data systems 110₁, 110₂ . . . 110_{N}, (or computer systems) accessible by one or more computing resources 120 across one or more networks 105. As illustrated, the different source data systems 110₁, 110₂ . . . 110_{N} can include, for instance, one or more medical records file systems 111₁, 111₂ . . . 111_{N}, each of which can contain electronic files of medical data records of one or more individuals. In the example illustrated, source data system 1 includes a medical records file system 111₁ with pathology-type electronic files 112 of multiple individuals, source data system 2 includes a medical records file system 111₂ with radiology-type electronic files 113, and clinical-type electronic files 114, relating to different individuals, and data source system N includes one or more medical records file systems 111_{N} with clinical-type electronic files 115 of different individuals. Note that this computing environment embodiment, and data source system example, represent one embodiment only of certain aspects disclosed herein, and that the electronic files discussed herein can reside on a variety of different systems, in one or more different configurations.

By way of example, network(s) 105 can be, for instance, a telecommunications network, a local-area network (LAN), a wide-area network (WAN), such as the Internet, or a combination thereof, and can include wired, wireless, fiber-optic connections, etc. The network(s) can include one or more wired and/or wireless networks that are capable of receiving and transmitting electronic data, including electronic pathology-type files, electronic radiology-type files, and electronic clinical-type files, as well as other electronic data, such as those discussed herein.

In one or more embodiments, computing resource(s) 120 can be a server-based resource, or a cloud-based computing resource, which includes program code 121 executing on one or more processors to implement one or more aspects of the electronic medical data records processing disclosed herein. In the embodiment illustrated, program code 121 implements, or has associated therewith, a cognitive engine 122, such as an artificial intelligence (AI)-based engine, which includes or provides one or more aspects of the electronic medical data records processing discussed herein including, for instance, a case abstraction generation component 123, and a case abstraction presentation/recommendation component 124. In one embodiment, program code 121, and in particular, cognitive engine 122, receives case input data 125, such as from one or more user-systems 140. In one embodiment, case input data 125 includes individual identifying data 126, and medical condition identifying data 127, identifying, for instance, a particular individual (or patient) and an associated medical condition for which electronic files are to be scanned and compiled into an electronic case abstraction, in accordance with one or more aspects disclosed herein.

In the embodiment of FIG. 1, computing environment 100 further includes one or more knowledge databases 130 accessible by, or part of, one or more computing resource(s) 120 for storing, in one embodiment, one or more electronic case abstractions 132. In one or more embodiments, the stored electronic case abstractions are accessible to cognitive engine 122 as a knowledge base for use as a reference, for example, by one or more machine learning models trained to make one or more predictions and/or recommendations, such as described herein.

Further, computing environment 100 includes, in one embodiment, one or more user systems 1 . . . N 140, such as one or more computing devices or computing systems, to which an electronic case abstraction compiled by cognitive engine 122 can be forwarded, for instance, across network(s) 105. In one or more implementations, a user system 140 can include, or have associated therewith, a case management application (or app) 142 and a display 144, as well as other computing device/system components. Case management application 142 is, in one or more embodiments, configured with a user interface to computing resource(s) 120 to facilitate the request for, and receipt and display of an electronic case abstraction, as well as, through appropriate “hooks”, the ability to access one or more of the compiled electronic medical data records of the individual related to the particular medical condition for display on the user system. In one or more embodiments, the compiled electronic case abstraction for a particular patient's medical condition contains links or hooks to the medical record files selected for inclusion in the case abstraction. For instance, the case management application includes (or accesses on computing resource(s) 120) viewers that load and present the compiled medical records through the hooks that the case abstraction embeds. Thus, in one or more implementations, the case management application allows for the presentation of the identified electronic medical data records of the electronic case abstraction at the user's system with reference to the embedded hooks, which can be, for instance, a web link, a database address, etc. Through an embedded hook or link it is possible to trigger the downloading and displaying of each identified electronic medical data record of the electronic case abstraction from where they are currently stored, or even from where originally stored.

By way of further example, user system(s) 140 can be a mobile computing system or resource, such as a smartphone, a wireless computer, a tablet, a personal digital assistant (PDA), a laptop computer, etc., or can be a desktop computer, server, or other computing system, with a case management application such as described herein configured to perform one or more aspects of the electronic case abstraction requesting, receiving, and/or displaying of records discussed herein. Note also that computing environment 100 can include additional and/or different systems, components, modules, subsystems, etc., without departing from the spirit of the present invention.

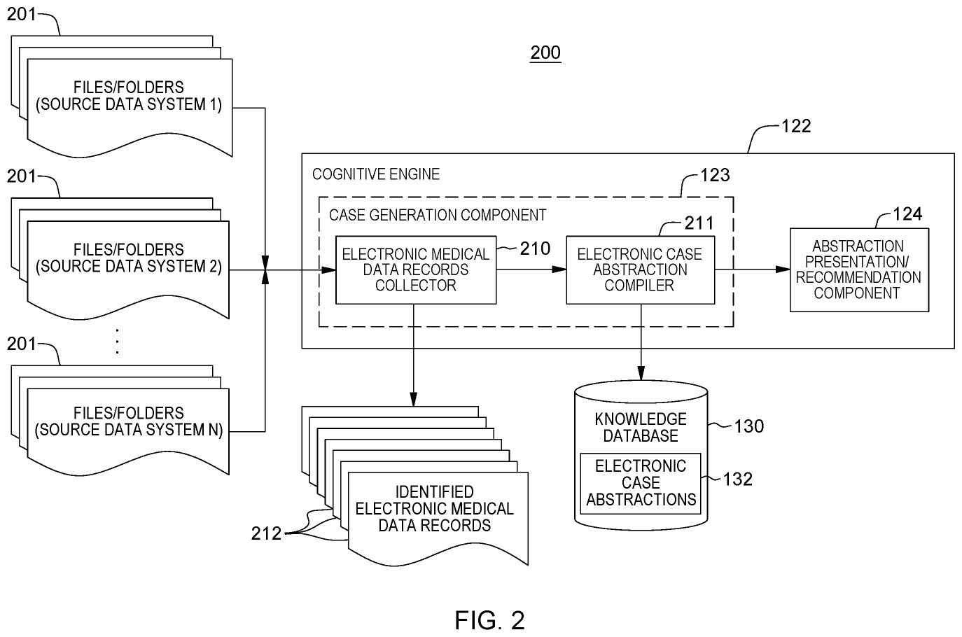

FIG. 2 depicts one embodiment of a computing environment 200 and workflow illustrating certain aspects of the present invention. In one or more implementations, computing environment 200 is similar to computing environment 100 described above in connection with FIG. 1, and includes, by way of example, multiple source data systems 1, 2 . . . N, with respective file systems containing a plurality of different types of electronic files/folders 201 containing medical data records of different individuals. Cognitive engine 122 is provided, in one or more implementations, on or in association with computing resources, such as described above, and includes a case generation component 123 and an abstraction presentation/recommendation component 124. In one embodiment, case generation component 123 can include an electronic data records collector 210, and an electronic case abstraction compiler 211.

In one or more embodiments, electronic data records collector 210 includes program code to electronically scan, by one or more processors, a set of electronic files to identify electronic medical data records 212 of an individual related to a specified medical condition. For instance, in one or more implementations, the individual's identity data is used to identify and obtain electronic medical data records for the particular patient identified. Further, depending on the specified medical condition, and using a reference database that serves as a learning source, the cognitive engine knows the types of medical records and intended diagnostics that are regularly considered when the specific health condition is being investigated. In one implementation, this knowledge base can be used, for instance, to provide a machine learning recommendation that certain record types are needed, either that are not currently available, or that the patient will need to undergo one or more tests or procedures to obtain the records. For excluding electronic files of the individual not related to the specified condition, the algorithm can electronically inspect each medical record looking for words that make the record not eligible for the particular electronic case abstraction. For instance, the condition may relate to a particular body part, and/or a particular type of exam can be used to filter records based on the condition. For example, pathology exams are not commonly required for a typical bone fracture.

As noted, the identified electronic medical data records 212 can include medical data records with different file types and different unique identifiers, and file records from different electronic medical records systems, such as different, incompatible electronic medical records systems. In one embodiment, electronic case abstraction compiler 211 includes program code configured to electronically compile the selected electronic medical data records of the individual related to the medical condition into an electronic case abstraction, with the compiling including associating a common case identifier to the electronic medical data records of the individual related to the medical condition.

In one or more implementations, the abstraction presentation/recommendation component 124 includes program code configured to electronically provide the case abstraction for display on a user system display screen in a consolidated view, to facilitate medical care of the individual. For instance, in one embodiment, the abstraction presentation/recommendation component 124 is configured to forward or transfer the electronic case abstraction to a requesting user system, via the case management application on the user system, for display on the user system, in order to facilitate a physician's or other specialist's review of the electronic case abstraction, and/or one or more of the electronic medical data records identified therein, and thereby assist in medical care of the individual.

In one or more implementations, a knowledge database 130 associated with cognitive engine 122 and/or accessible by cognitive engine 122, stores the electronic case abstraction with other electronic case abstractions 132 for further reference, in accordance with one or more aspects, in providing machine learning-based recommendations related to one or more patient conditions.

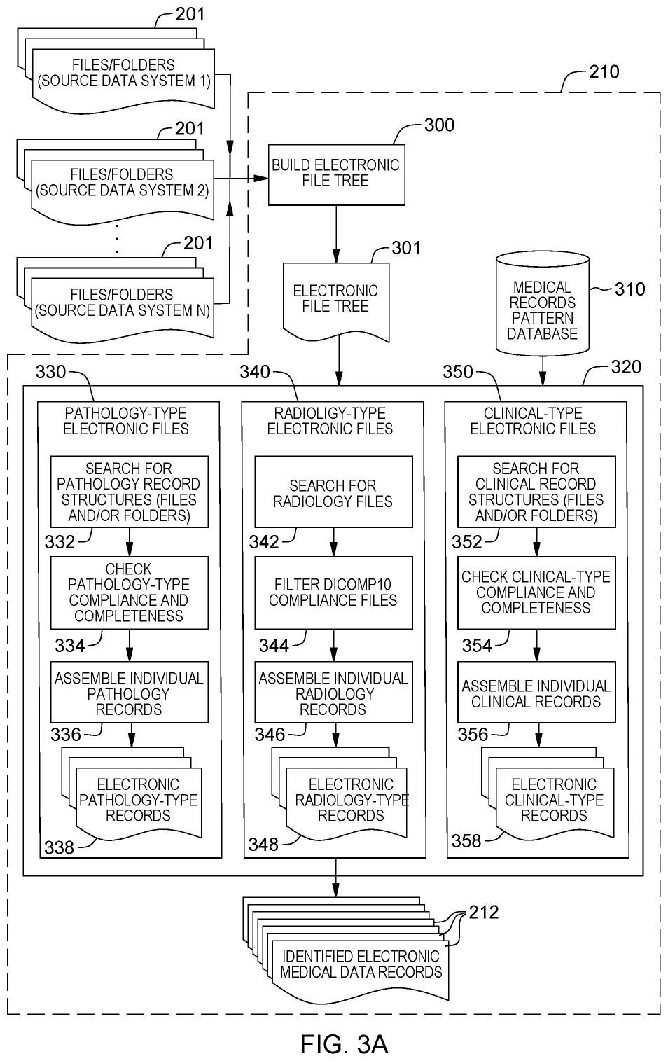

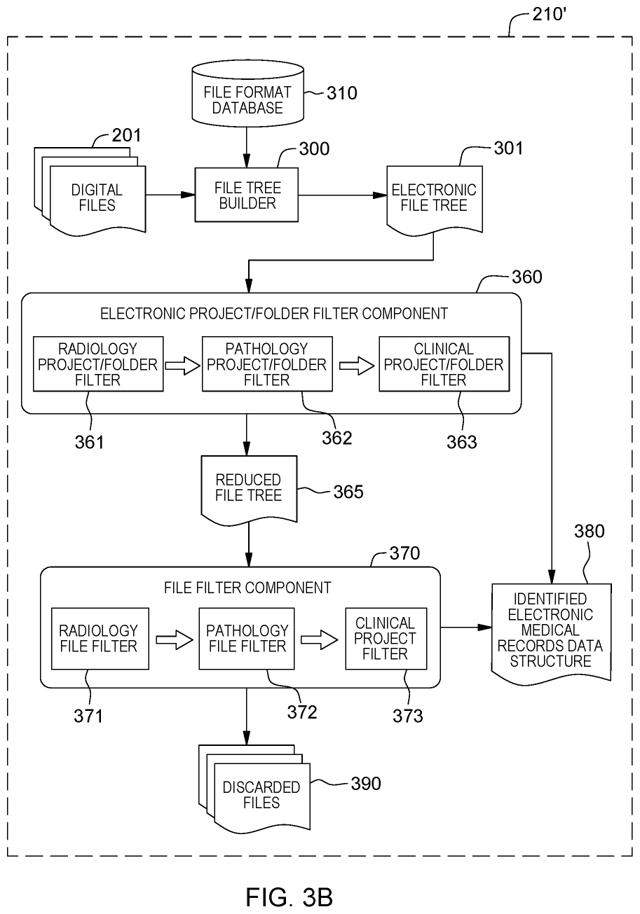

FIG. 3A depicts one embodiment of a collector workflow illustrating certain aspects of one or more embodiments of the present invention. In one embodiment, electronic data records collector 210 includes program code configured to build 300 an electronic file tree 301 containing a set of potentially relevant electronic files obtained from files/folders 201 dispersed across one or more different electronic medical records systems (e.g., source data system 1, source data system 2, . . . source data system N). The electronic files/folders 201 provided as input for building 302 electronic file tree 301 can be loaded or accessed in a variety of ways. For instance, a drag and drop feature can be provided on the user systems to enable a user to provide the cognitive engine with access to certain electronic files at issue, or the location of one or more files/folders can be identified on storage media, such as on different source data systems. In one implementation, a heuristic algorithm is generated which is configured to detect applicable electronic medical data records from a variety of data collections (i.e., different files and folders), which serve as the input for the cognitive engine algorithm. Note in this regard that records can be collected from any number of data source systems concurrently, in one embodiment.

Electronic data records collector 210 includes, and/or utilizes, in one or more embodiments, a medical records pattern database 310 of known medical data record patterns, such as known pathology record structures, known radiology record structures, and known clinical record structures. In one implementation, the medical records pattern database 310 can be dynamically updated as the cognitive engine performs automated scanning of file system volumes in order to detect 320 the structures (i.e., files and/or folders) that are potentially part of medical records of an individual for a particular condition. Note that as used herein, an electronic folder can include multiple electronic files, and an electronic medical record can have one or more electronic records and/or electronics files. In one or more embodiments, three distinct classes of medical records, or medical records units, are detectable, including pathology-type electronic files 330, radiology-type electronic files 340, and clinical-type electronic files 350. For instance, the cognitive engine searches the electronic files and folders for pathology records structures 332. The cognitive engine also checks pathology-type compliance and completeness 334, and assembles or builds the electronic files into individual pathology records or units 336, which are provided as electronic pathology records 338 (e.g., slides), to form part of the identified electronic medical data records 212. Similarly, the radiology-type electronic files 340 are searched for radiology files 342, which are filtered for DICOM compliance files 344. The resulting files are assembled or built into individual radiology records or units 346, and provided as electronic radiology records (e.g., studies) 348 for inclusion in identified electronic medical data records 212. Further, the clinical-type electronic files 350 are identified by searching the files/folders of the electronic file tree for clinical records structures 352. Any clinical records structures are checked for clinical-type compliance and completeness 354 before being assembled as individual clinical records or units 356 into the electronic clinical data records (e.g., documents) 358, which are provided as part of identified electronic medical data records 212. For each file type, the structure of the file is thus validated in terms of format compliance and completeness to ensure that all electronic medical data records identified can be properly cataloged and presented in association with the electronic case abstraction being compiled.

FIG. 3B depicts an alternate embodiment of an electronics data records collector 210′. Electronic medical data records collector 210′ includes program code implementing a heuristic data processing algorithm that executes to scan and classify medical records from different digital file sources, and link them to the electronic case abstraction by adding identified entries, in one embodiment, to a records list or records data structure. In one or more implementations, the electronic medical data records collector 210′ is cognitive engine-implemented processing which receives as input a set of digital electronic files 201 (and/or folders), and through a heuristic scan process, traverses the set of electronic files in order to detect and classify which electronic medical data records of the individual are to be added to the electronic case abstraction. Note that a single medical record can be represented by one or more digital files, and that there are at least three principal types of medical records that the cognitive engine detects and extracts data from, such as metadata. These are, in one implementation, pathology files (which include pathology digital slides and pathology report files), radiology files (which include radiology images and radiology report files), and clinical reports (which include clinical exams).

As illustrated in FIG. 3B, the heuristic processing for detecting the different types of relevant medical records, initially builds via a file tree builder 300, with reference to a file format database 310, an electronic file tree 301 containing selected electronic digital files to be further scanned for possible identification and/or inclusion in the electronic case abstraction.

Note that building the electronic file tree as described herein is one embodiment only for collecting and associating electronic files to be evaluated more closely for inclusion in an electronic case abstraction, such as described. In one embodiment, as part of building the file tree, each inputted electronic file can be assigned a tag that defines its file type. There are files that conventionally do not have a file type detected from its extension, and content processing can be performed to discover the particular file format. A file format database can be included to provide file type signatures in order to facilitate this process. Once the electronic file tree 301 is built, multiple levels of filtering can be executed in the workflow embodiment of FIG. 3B. First, an electronic project/folder filter component 360 can be executed to identify medical records that are organized as projects or folders. Based on specific folder structure and signature of files, the cognitive engine can identify what files within specific folders are part of a single medical record. The files that are classified as part of a medical record are removed from the file tree and grouped as part of the identified electronic medical data records list, with the reduced file tree 365 being further processed. In the embodiment of FIG. 3B, the electronic project/folder filtering includes a radiology project/folder filter 361, a pathology project/folder filter 362, and a clinical project/folder filter 363, each configured to identify the respective type of folder.

In the embodiment of FIG. 3B, the reduced file tree 365 is provided to an electronic files filter component 370, which includes program code configured to detect whether each remaining file is a single-file medical record, or part of a multi-file medical record. There are medical data records that are built from multiple electronic files, which are detectable based on metadata present in their content. By processing each file's metadata, the algorithm is able to group which files are part of a single medical data record, with electronic files not classified as medical data records being discarded 390. In the embodiment illustrated, electronic file filter component 370 includes a radiology file filter 371, a pathology file filter 372, and a clinical project file filter 373. Once identified, the electronic medical data records are listed or retained as part of the identified electronic medical data records data structure 380.

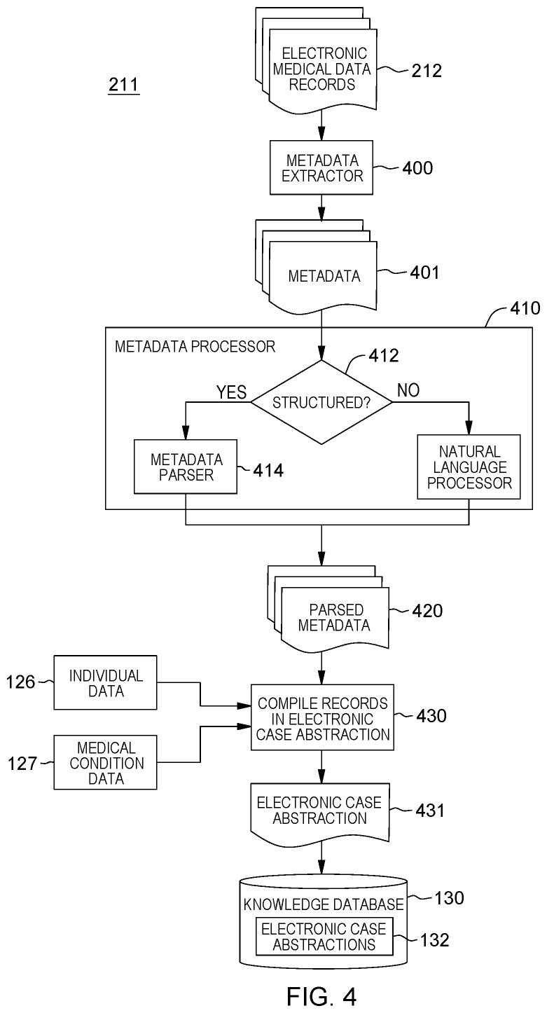

FIG. 4 depicts one embodiment of a compiler workflow illustrating certain aspects of one or more embodiments of the present invention. As illustrated, in one or more embodiments, the electronic case abstraction compiler includes program code configured to implement, for instance, a cataloging algorithm to extract and update metadata from the identified electronic medical data records 212 in order to consolidate the records as part of a particular electronic case abstraction for a specific patient's condition. The compiler is configured with the ability to build a case abstraction that connects medical data records and their metadata to a particular patient, and to a specific health condition context that needs to be medically addressed. In one embodiment, the electronic case abstraction is provided by a self-sufficient program code module or unit configured to aggregate information required by a health care professional in a diagnostic workflow context.

As illustrated in FIG. 4, in one embodiment, the identified electronic medical data records 212 are obtained by the compiler 211, which includes a metadata extractor 400 to extract metadata 401 from each electronic medical data record. A metadata processor 410 is associated with the compiler for determining whether metadata associated with a particular electronic medical data record is structured 412. If “yes”, then a metadata parser 414 obtains parsed metadata 420 from the structured metadata for the electronic medical data record. Otherwise, a natural language processor 416 parses the unstructured metadata of the electronic medical data record to obtain the parsed metadata 420. The parsed metadata 420 provides file grouping information, and is combined with identifying patient data 126, as well as, for instance, context-specific case data 127 (i.e., the relevant medical condition) in order to tag or assign a common case identifier to each identified electronic medical data record. Once tagged or assigned a common case identifier, the electronic medical data record becomes a component of the electronic case abstraction 431. In one embodiment, a copy of the electronic case abstraction 431 is saved to knowledge database 130, along with one or more other electronic case abstractions 132 for, for instance, subsequent analysis and/or retrieval.

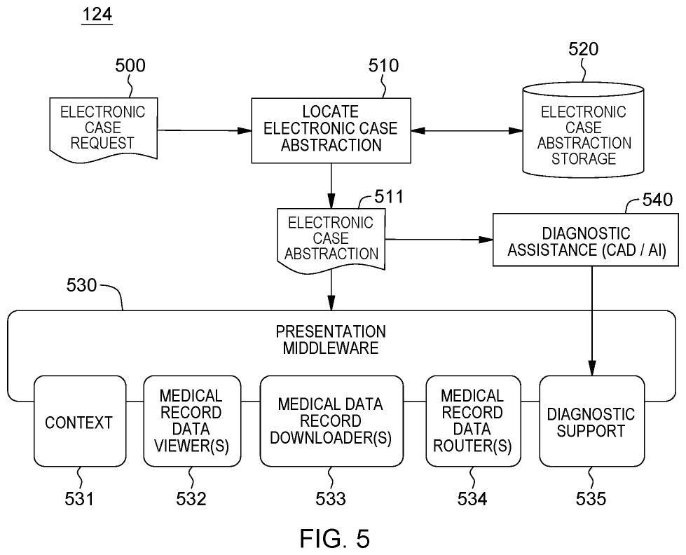

FIG. 5 illustrates one embodiment of a presentation/recommendation component 124 workflow depicting certain aspects of one or more embodiments of the present invention. In one or more implementations, the workflow is implemented by program code configured, at least in part, to perform one or more aspects of electronically providing, by one or more processors, for display on a display screen in a consolidated view, the electronic case abstraction to facilitate medical care of the individual, such as described herein. For instance, uploaded electronic files are presented in a categorized list in the electronic case abstraction, which can be selected by clicking on the file name, and then automatically displayed using an appropriate display application. For example, CCDA can be used for EHR extracted data files, a PDF viewer for text files, a DICOM viewer for radiology files, and a pathology viewer for pathology images. In one or more embodiments, file categories can be changed by the user for their convenience. For example, if a radiology report is embodied in a PDF file that cannot be otherwise discerned, then the report can be moved (e.g., dragged) from a clinical category to a radiology category for viewing.

In the embodiment of FIG. 5, an electronic case request 500 is received and the requested electronic case abstraction is located 510, for instance, from a knowledge base or other electronic case abstraction storage 520. Note that if the requested electronic case abstraction is yet to be compiled, then processing such as described herein proceeds to generate the requested electronic case abstraction. The electronic case abstraction 511 is forwarded to presentation middleware 530 to facilitate downloading and/or presentation of particular electronic medical data records in the case abstraction. In one or more implementations, presentation middleware 530 embeds Vendor Neutral Archive (VNA) and/or Electronic Medical Records (EMR) adapters in viewer launchers required to display electronic medical data records of different file types and formats, such as described herein. The VNA adapters are connectors that enable the cognitive engine to interconnect to systems from where medical record files can be retrieved to be processed to be part of an electronic case abstraction, or to systems that are prepared to receive the electronic case abstraction to be stored and/or presented. As noted, medical data records can be stored in a variety of locations and/or systems, and referenced within the electronic case abstraction via a hook, such as a web link, a database address, etc. Through the hook, it is possible to trigger the download and visualization of one or more electronic medical data records of the electronic case abstraction from where they are currently stored, or even originally stored. Viewer launchers are also adapters that enable dedicated viewing applications to be launched from the electronic case abstraction. The viewer application will receive the location where the medical record is stored, retrieve it, and present it for display on the user system. In one implementation, the presentation middleware includes context middleware 531, medical record data viewer(s) middleware 532, medical data record downloader(s) middleware 533, medical record router(s) middleware 534, and diagnostic support middleware 535. Note that the depicted middleware in this context is embedded code configured to facilitate one or more aspects disclosed herein.

In FIG. 5, the abstracted presentation/recommendation component 124 further includes, by way of example, diagnostic assistance code 540, such as a computer-aided diagnostic (CAD) system or artificial intelligence (AI)-implemented engine that relies on the electronic case abstraction knowledge database to be continually informed from usage, and to provide guidance and analytic data on how similar medical condition cases were previously handled, and to present the current electronic case abstraction in an appropriate manner to assist in medical care of the individual.

Advantageously, the electronic case abstraction, and supporting cognitive engine and system, provides a universal access interface that enables the presentation of electronic case abstractions and the associated electronic medical data records to third party systems, such as third party user systems. Upon receiving an access request for an existing electronic case abstraction, the cognitive engine renders back the context for which the electronic case abstraction was originally created to provide access to all of the electronic medical data records identified and attached to that case abstraction. Advantageously, medical data records of any format can thus be presented in a single combined interface, where they can be viewed, downloaded or routed to other systems.

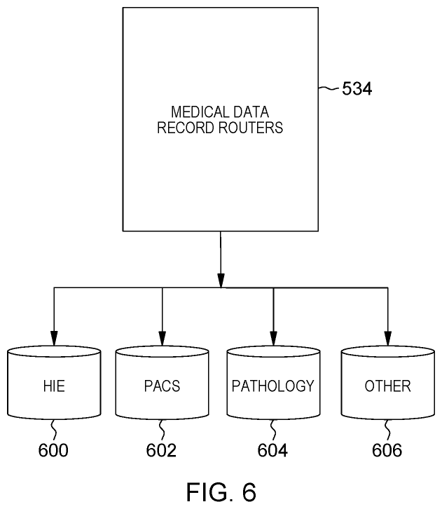

By way of further example, FIG. 6 illustrates one embodiment of medical data record routers 534 for routing medical data records to different destinations based on file type. As discussed herein, electronic medical data records associated with an electronic case abstraction can be stored, in one embodiment, to the appropriate data storage based on file or record type. Routing rules can be imposed, where the intermediary may approve a record prior to its routing. Alternatively, electronic files may be directly routed per set rules. For instance, medical data record routers 534 can route appropriate data to an electronic Health Information Exchange (HIE) 600, which allows electronic sharing of patient data in a secure manner, to a Picture Archiving and Communication System (PACS) 602 for storing medical imaging data, to a pathology file system, for storing pathology-type files 604, or other file system 606.

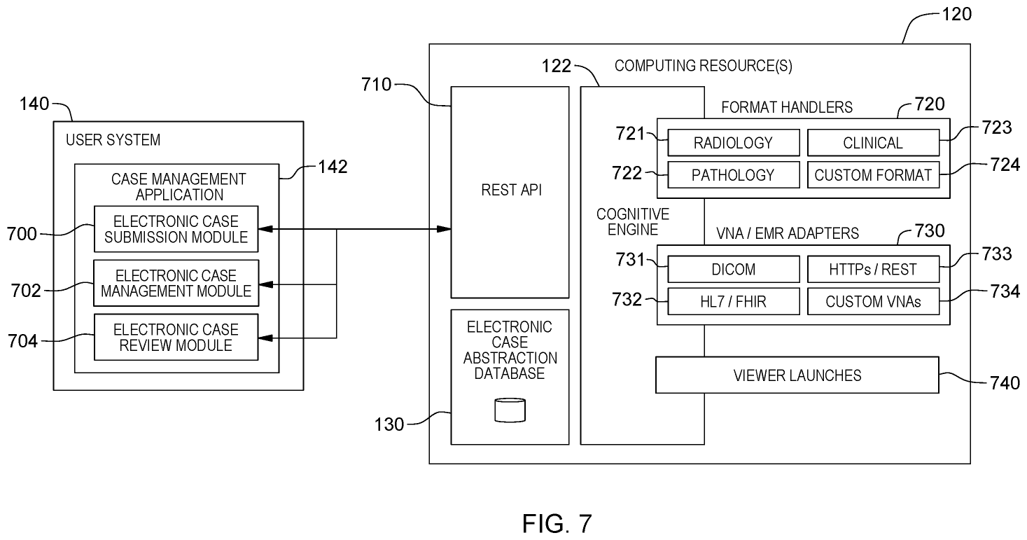

FIG. 7 depicts another example of a computing environment to incorporate and use one or more aspects of the present invention. The depicted computing environment is similar to computing environment 100 described above in connection with FIG. 1, and includes (for instance) one or more computing resources 120, and one or more user systems 140. As noted, user system 140 includes, in one embodiment, a case management application or system 142, such as described herein, which facilitates handling the lifecycle of each electronic case abstraction created in order to facilitate addressing a specific health condition. The computer-implemented methods, computer systems and computer program products disclosed embody human system interfaces that enable the interaction of a stakeholder (e.g., patient, caregiver, physician, other expert) with an electronic case abstraction related to a particular medical condition of the patient as it evolves. As illustrated, the case management application 142 interfaces with cognitive engine 122 via one or more application programming interfaces, such as a representational state transfer (REST) API 710. Case management application 142 can retrieve electronic case abstractions and visually present them, in one embodiment, via an Internet browser, giving the end-user the ability to read textual information and visualize medical records encapsulated into the electronic case abstraction.

By way of example, in FIG. 7, case management application 142 includes an electronic case submission module 700, which includes, in one embodiment, an end-user interface dedicated to initiating a new electronic case abstraction, including, for instance, providing case input data, such as individual identifying data and medical condition identifying data to be used in compiling or identifying the electronic case abstraction. Further, case management application 142 includes an electronic case management module 702, which includes program code configured as a user interface for monitoring the electronic case abstraction lifecycle creation, including while medical records are being aggregated, and when the electronic case abstraction is being reviewed. Further, case management application 142 includes, in one embodiment, an electronic case review module 704, which includes program code configured to provide an end-user interface for initiating creation of a case review, such as a diagnostic or other recommendation, to facilitate medical care of the individual for a particular medical condition. In one embodiment, the case management application 142 facilitates creating an electronic case abstraction containing the patient's medical condition, and the cognitive engine 122 executing on computing resource(s) 120 is configured to populate the electronic case abstraction with electronic medical data records of the individual related to the condition, plus in one or more embodiments, diagnostic and/or other patient information. In one or more implementations, once created, the electronic case abstraction is reviewed, such as by an end-user via user system 140, and uploaded into a database, such as a knowledge database 130 containing a plurality of electronic case abstractions.

As described herein, cognitive engine 122 includes, in one embodiment, an algorithmic engine or artificial intelligence-based engine, that handles collection, compilation and presentation of electronic medical data records, such as described. Functional modules of the computing environment of FIG. 7 include, by way of example, REST API 710, which implements an HTTP web API that enables, for instance, create, read, update and delete operations for or on electronic case abstractions, such as stored in electronic case knowledge database 130. Electronic case knowledge database 130 holds, in one embodiment, electronic case abstractions and is, for instance, a relational database that stores complex objects, such as objects of JavaScript Object Notation (JSON) type.

Computing resource(s) 120 includes format handlers 720, which are, in one embodiment, specific program code routines that are used to receive, for instance, a file tree and scan the file tree looking for electronic medical data records of an individual of a particular class, such as radiology 721, pathology 722, clinical 723, or other custom format 724. The identified files are grouped, and any single medical data record at the end of processing is removed from the file tree.

In one embodiment, collecting electronic medical data records (e.g., digital files) can be facilitated via a set of adapters that handle the interoperability with other computing systems, including vendor neutral archives (VNAs) and electronic health records (EHR) 730, from where electronic medical data files or records are obtained including, for instance, via, for instance, DICOM adapters 731, HL7/FHIR adapters 732, HTTP/REST adapters 733, and available custom VNA adapters 734.

Cognitive engine 122 also includes or is in communication with viewer launchers 740, which are (in one embodiment) program code components that are used to launch external software-based views that will load and present electronic medical data records that are added to the electronic case abstraction. For a medical record class, a dedicated viewer is launched, receiving a list of parameters defining from where the electronic medical data record to be visualized can be loaded from. Note that the viewers can be, in one or more embodiments, open source components or licensed third-party viewers, that are integrated into or embedded within program code such as disclosed herein.

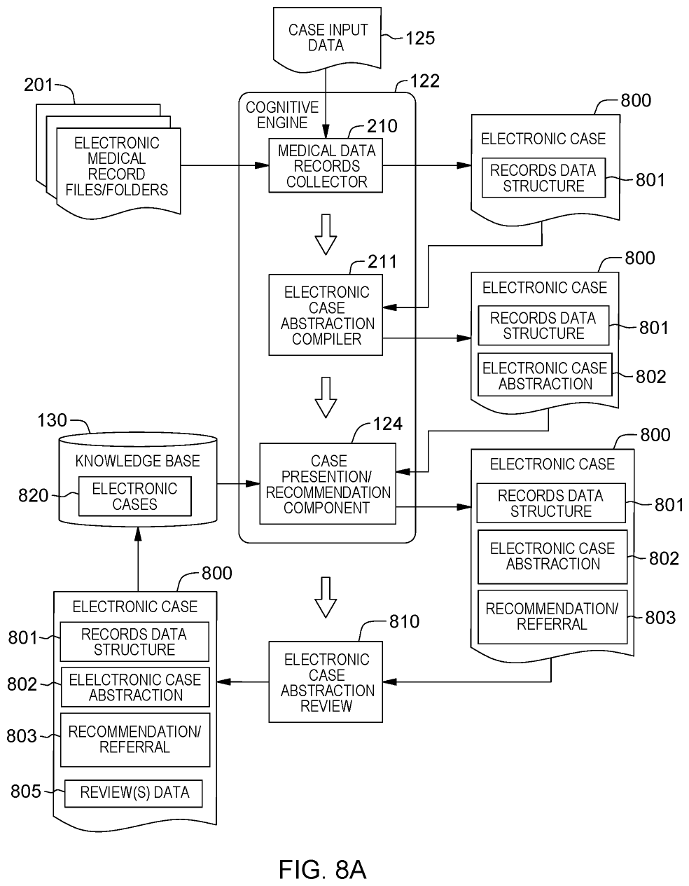

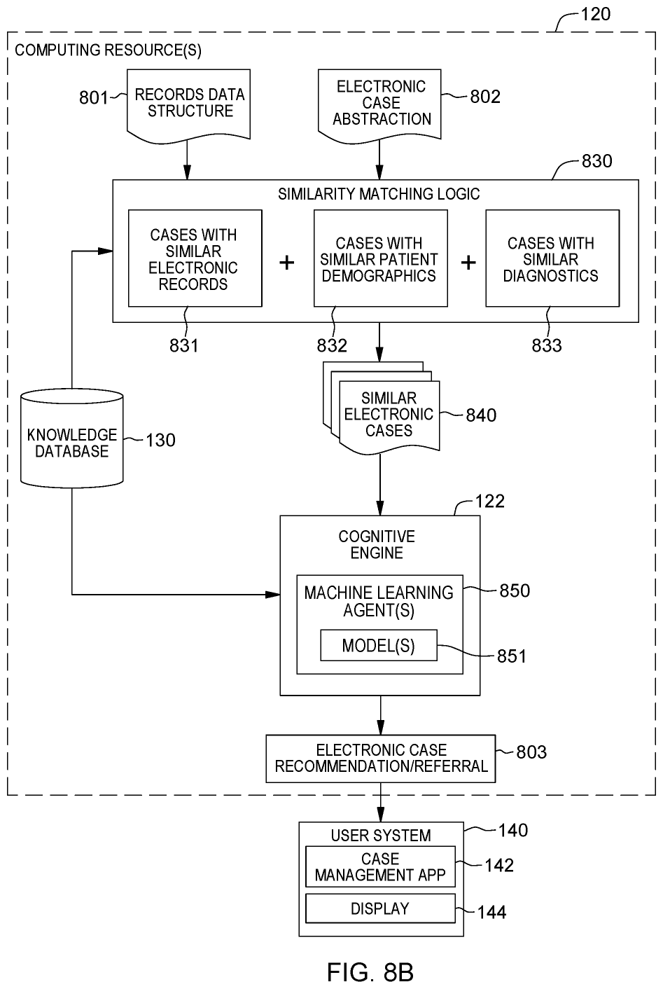

FIG. 8A depicts another embodiment of a workflow illustrating certain aspects of one or more embodiments of the present invention. In particular, FIG. 8A depicts further details of one embodiment of generating an electronic case 800 for facilitating medical of an individual related to a particular medical condition. As explained above in connection with FIGS. 1-7, in one or more implementations, a cognitive engine 122 receives case input data 125 and obtains electronic record files/folders 201 for collecting, via medical data records collector 210, identified electronic medical records in a records data structure or list 801 of electronic case 800. The records data structure 801 operates, in one or more embodiments, as an aggregation unit, maintaining references to medical records that are relevant to the electronic case abstraction being compiled. In one or more implementations, medical data records collector 210 scans the medical record files/folders and classifies the medical records from the digital files, linking selected records to the case abstraction by adding entries to the records data structure 801. Electronic case abstraction compiler 211 builds the electronic case abstraction 802 by linking the identified medical records, using metadata parsing and natural language processing, to build the electronic case abstraction 802 with a common case identifier. In one embodiment, the electronic case abstraction 802 can include, for instance, a patient data structure and a diagnostics data structure. The patient data structure can include patient demographic information and the unique identifiers assigned to the patient by the data source systems, or file systems, where the electronic medical record was generated. The diagnostics data structure can hold, in one embodiment, diagnostic information extracted from the medical records linked to the electronic case abstraction.

As an electronic case abstraction is reviewed 810, the review data 805 can be added to the electronic case 800, which can then be added to an electronic cases 820 knowledge database 130, and used, in one embodiment, to feed an artificial intelligence (AI) engine, such as on one or more machine learning models, that capture and learn the specialists and treatments involved in each particular medical condition. Once a new medical condition is created or updated with medical records, the case presentation/recommendation component 124 is configured to look for similarities in reviewed cases in order to infer or predict a recommendation or referral, for instance, to suggest what additional specialist or treatment should be considered. The recommendation/referral 803 can be added as part of the electronic case 800 being reviewed 810.

FIG. 8B depicts further details of one embodiment of a computing environment and workflow illustrating certain aspects of one or more embodiments of the present invention. The computing environment and workflow illustrated are similar to those described above in connection with FIGS. 1-8A, with the addition of one or more machine learning agents 850 and associated machine learning models 851 to facilitate electronic case recommendation/referral 803, such as described herein. As depicted, computing resource(s) 120, and more particularly, the cognitive engine thereof, can include program code executing on one or more processors to implement similarity matching logic 830. In one embodiment, similarity matching logic 830 receives the records data structure 801 and the electronic case abstraction 802 of the particular electronic case, and with reference to knowledge database 130, scans for cases with similar electronic records 831, such as a similar set of electronic records, scans for cases with similar patient demographics 832, and scans for cases with similar diagnostics 833, such as similar exam results. The result is an output of similar electronic cases 840, which the cognitive engine 122 processes using a trained machine learning model(s) 851 to generate predictions and/or recommendations, such as electronic case recommendation/referral 803, which can be provided as part of the electronic case to a user system 140 via the case management application 142 for display 144.

FIG. 9 depicts a further embodiment of a computing environment or system 900, incorporating, or implementing, certain aspects of an embodiment of the present invention. In one or more implementations, system 900 can be part of a computing environment, such as computing environment 100 described above in connection with FIG. 1. System 900 includes one or more computing resources 910 that execute program code 912 that implements, for instance, an electronic case recommendation/referral facility, and includes a cognitive engine 914, which has one or more machine-learning agents 916, and one or more machine-learning models 918. Data 920, such as the different data discussed herein, is used by cognitive engine 914, to train model(s) 918, to (for instance) predict a medical diagnosis and to generate one or more recommendations or actions 930, etc., based on the particular application of the machine-learning model. In implementation, system 900 can include, or utilize, one or more networks for interfacing various aspects of computing resource(s) 910, as well as one or more data sources providing data 920, and one or more user systems receiving the electronic case abstraction and/or recommendation, referral, etc., 930 of machine-learning model(s) 918. By way of example, the network(s) can be, for instance, a telecommunications network, a local-area network (LAN), a wide-area network (WAN), such as the Internet, or a combination thereof, and can include wired, wireless, fiber-optic connections, etc. The network(s) can include one or more wired and/or wireless networks that are capable of receiving and transmitting data, including training data for the machine-learning model, predicted traffic event and an output solution, recommendation, action, of the machine-learning model, such as discussed herein.

In one or more implementations, computing resource(s) 910 houses and/or executes program code 912 configured to perform methods in accordance with one or more aspects of the present invention. By way of example, computing resource(s) 910 can be a computing-system-implemented resource(s). Further, for illustrative purposes only, computing resource(s) 910 in FIG. 9 is depicted as being a single computing resource. This is a non-limiting example of an implementation. In one or more other implementations, computing resource(s) 910, by which one or more aspects of machine-learning processing such as discussed herein are implemented, could, at least in part, be implemented in multiple separate computing resources or systems, such as one or more computing resources of a cloud-hosting environment, by way of example.

Briefly described, in one embodiment, computing resource(s) 910 can include one or more processors, for instance, central processing units (CPUs). Also, the processor(s) can include functional components used in the integration of program code, such as functional components to fetch program code from locations in such as cache or main memory, decode program code, and execute program code, access memory for instruction execution, and write results of the executed instructions or code. The processor(s) can also include a register(s) to be used by one or more of the functional components. In one or more embodiments, the computing resource(s) can include memory, input/output, a network interface, and storage, which can include and/or access, one or more other computing resources and/or databases, as required to implement the machine-learning processing described herein. The components of the respective computing resource(s) can be coupled to each other via one or more buses and/or other connections. Bus connections can be one or more of any of several types of bus structures, including a memory bus or a memory controller, a peripheral bus, an accelerated graphics port, and a processor or local bus, using any of a variety of architectures. By way of example, but not limitation, such architectures can include the Industry Standard Architecture (ISA), the micro-channel architecture (MCA), the enhanced ISA (EISA), the Video Electronic Standard Association (VESA), local bus, and peripheral component interconnect (PCI). As noted, examples of a computing resource(s) or a computer system(s) which can implement one or more aspects disclosed herein are described further herein with reference to FIG. 1, as well as with reference to FIG. 10.

Program code 912 executes, in one implementation, a cognitive engine 914 which includes one or more machine-learning agents 916 that facilitate training one or more machine-learning models 918. The machine-learning models are trained using training data that can include a variety of types of data, depending on the model and the data sources. In one or more embodiments, program code 912 executing on one or more computing resources 910 applies machine-learning algorithms of machine-learning agent 916 to generate and train the model(s), which the program code then utilizes to predict, for instance, a recommendation or referral, and depending on the application, to perform an action (e.g., provide a solution). In an initialization or learning stage, program code 912 trains one or more machine-learning models 918 using obtained training data that can include, in one or more embodiments, medical data records, such as described herein.

Training data used to train the model (in embodiments of the present invention) can include a variety of types of data, such as data generated by or obtained from one or more file system components or utilities, in communication with the computing resource(s). Program code, in embodiments of the present invention, can perform machine-learning analysis to generate data structures, including algorithms utilized by the program code to predict and/or perform a machine-learning action. As known, machine-learning (ML) solves problems that cannot be solved by numerical means alone. In this ML-based example, program code extract features/attributes from training data, which can be stored in memory or one or more databases. The extracted features are utilized to develop a predictor function, h(x), also referred to as a hypothesis, which the program code utilizes as a machine-learning model. In identifying machine-learning model 918, various techniques can be used to select features (elements, patterns, attributes, etc.), including but not limited to, diffusion mapping, principal component analysis, recursive feature elimination (a brute force approach to selecting features), and/or a random forest, to select the attributes related to the particular model. Program code can utilize a machine-learning algorithm to train machine-learning model (e.g., the algorithms utilized by program code), including providing weights for conclusions, so that the program code can train any predictor or performance functions included in the machine-learning model. The conclusions can be evaluated by a quality metric. By selecting a diverse set of training data, the program code trains the machine-learning model to identify and weight various attributes (e.g., features, patterns) that correlate to enhanced performance of the machine-learned model.

Some embodiments of the present invention can utilize IBM Watson® as learning agent. IBM Watson® is a registered trademark of International Business Machines Corporation, Armonk, N.Y., USA in one or more jurisdictions. In embodiments of the present invention, the respective program code can interface with IBM Watson® application program interfaces (APIs) to perform machine-learning analysis of obtained data. In some embodiments of the present invention, the respective program code can interface with the application programming interfaces (APIs) that are part of a known machine-learning agent to determine impacts of data on the machine-learning model, and to update the model, accordingly. In one or more embodiments, program code of the present invention can utilize and/or tie together multiple existing artificial intelligence (AI) applications.

In one or more embodiments of the present invention, the program code can utilize a variety of AI techniques, such as noted herein, and/or a neural network to analyze training data and/or collected data to generate an operational machine-learning model.

Neural networks are a programming paradigm which enable a computer to learn from observational data. This learning is referred to as deep learning, which is a set of techniques for learning in neural networks. Neural networks, including modular neural networks, are capable of pattern (e.g., state) recognition with speed, accuracy, and efficiency, in situations where datasets are mutual and expansive, including across a distributed network, including but not limited to, cloud computing systems. Modern neural networks are non-linear statistical data modeling tools. They are usually used to model complex relationships between inputs and outputs, or to identify patterns (e.g., states) in data (i.e., neural networks are non-linear statistical data modeling or decision-making tools). In general, program code utilizing neural networks can model complex relationships between inputs and outputs and identified patterns in data. Because of the speed and efficiency of neural networks, especially when parsing multiple complex datasets, neural networks and deep learning provide solutions to many problems in multi-source processing, which program code, in embodiments of the present invention, can utilize in implementing a machine-learning model, such as described herein.

Advantageously, disclosed herein are computer-implemented methods, computer systems and computer program products for facilitating patient medical care by remotely obtaining, compiling and presenting electronic medical data records of any type and format, from different electronic medical data records, in a single, case-dependent electronic abstraction. The facilities disclosed herein provide efficient electronic transfer of electronic medical records for a specified medical condition between locations using, for instance, different medical data record viewers, and different storage file systems. The differently-formatted medical data records can be consolidated into a single display for reference by a medical professional. Advantageously, from a browser environment, the medical professional is able to retrieve and review the entire set of patient medical data records on the particular condition, irrespective of the format of the data records. No technical integration between disparate, incompatible file systems or computer systems is required.

Embodiments of the present invention include program code executing on one or more processors which electronically scans a set of electronic files for electronic medical data records of an individual related to a specific medical condition. The scanning identifies electronic medical data records of the individual related to the medical condition which include different file types with different unique identifiers, and which are from different electronic medical records systems. Further, program code executing on one or more processors electronically compiles the identified electronic medical data records of the individual related to the medical condition into an electronic case abstraction, including associating a common case identifier with the identified medical data records of the individual related to the medical condition. In addition, program code executing on one or more processors electronically provides for display on a display screen in a consolidated view, the electronic case abstraction to facilitate medical care of the individual.

In one or more embodiments, the identified electronic medical data records of the individual related to the medical condition compiled for the electronic case abstraction of different file types include two or more of an electronic pathology-type file, an electronic radiology-type file, and an electronic clinical-type file.

In one embodiment, the scanning includes electronically scanning the set of electronic files using a heuristic algorithm and a database of known medical record patterns, to identify the electronic medical data records of the individual related to the medical condition which include different file types with different unique identifiers, and which are from different electronic medical records systems.

In one or more embodiments, the computer-implemented method further includes obtaining, by the one or more processors, the set of electronic files from the different electronic medical records systems, wherein two or more electronic medical records systems of the different electronic medical records systems are incompatible electronic medical records systems.

In one or more embodiments, the computer-implemented method further includes categorizing by file type and file context the identified electronic medical data records of the individual related to the medical condition, where categorizing by file context results in associating two or more identified electronic medical data records of different file type together in the electronic case abstraction. In one embodiment, the categorizing includes comparing electronic files of the set of electronic files to known file structures, including known pathology record structures, known radiology record structures, and known clinical record structures, to facilitate categorizing the identified electronic medical data records by file type. In one or more embodiments, the computer-implemented method further includes rules-based forwarding of the identified electronic medical data records to data storage in accordance with respective categorized file types of the identified electronic medical data records.

In one or more embodiments, the electronically compiling, by the one or more processors, the identified electronic medical data records of the individual related to the medical condition into the electronic case abstraction includes electronically compiling, by the one or more processors, the electronic case abstraction by extracting metadata from the identified electronic medical data records of the individual related to the medical condition, and processing the metadata to facilitate the electronically compiling of the electronic case abstraction, where extracting the metadata includes parsing structured metadata associated with one or more electronic medical data records of the identified electronic medical data records.

In one or more embodiments, the method further includes using a natural language processor to extract unstructured metadata from one or more other electronic medical data records of the identified electronic medical data records to obtain parsed metadata for the other electronic medical data record(s) for use in electronically compiling the electronic case abstraction.

In one or more embodiments, program code is provided to further train one or more machine learning models to make one or more recommendations to facilitate medical care of individuals based on electronic case abstractions, and to electronically provide, by the one or more processors, using the machine learning model(s), a recommendation to facilitate medical care of the individual related to the medical condition. In one embodiment, the electronically providing includes electronically providing, by the one or more processors, using the machine learning model(s), a medical referral for the individual related to the medical condition to a specialist.

Further, embodiments of the present invention include training one or more machine learning models to make one or more recommendations to facilitate medical care of individuals based on electronic case abstractions. Further, embodiments of the present invention include program code executing on one or more processors which electronically scans a set of electronic files for electronic medical records of an individual related to a specified medical condition. The scanning identifies electronic medical data records of the individual related to the medical condition, and which include different file types with different unique identifiers, and which are from different electronic medical records systems. Further, program code executing on one or more processors electronically compiles the identified electronic medical data records of the individual related to the medical condition into an electronic case abstraction, including associating a common case identifier with the identified medical data records of the individual related to the medical condition. In addition, program code executing on one or more processors electronically provides, using the machine learning model(s) a recommendation to facilitate medical care of the individual related to the medical condition.

Embodiments disclosed herein advantageously include computer-implemented methods, computer systems and computer program products which facilitate electronic transfer of medical records of any format between different entities in different locations. For instance, in one embodiment, a patient can electronically transfer medical records to a remote physician by, for instance, the physician hosting an uploader on a patient portal or a website, or sending a link to a patient. The patient can identify which electronic files, folders, etc., containing medical records, for instance, via address location on a computer or storage, or a drag-and-drop of files, folders, etc.

In certain embodiments, the present invention is related to electronically transferring medical data records between different locations, where the computer-implemented method, computer system and computer program product include a user-interface, implemented (for instance) using an Internet browser, that is published at a specified URL by the receiving location. The sender can identify medical record files to be transferred by locating the files on a storage device, or by dragging and dropping the files to a specific location in the sender's screen. The connection between the two systems can be established as a secure socket layer, with the files sent and deposited into the receiving system's storage.

In one or more embodiments, component parts of one server application implementation include uploader software to enable file uploads of any format, for instance, using a standard browser on a computer, or application on a mobile device, a records tagging and case identification system, a records organizer subsystem, a router subsystem, an audit subsystem, a malware scanner subsystem, a storage subsystem, VNA adapter subsystem, medical records viewer subsystems, HL7 connectors to both a third-party user interface for patients, and a third-part interface for clinicians, and a messaging subsystem.

In one embodiment, the uploader contains a graphic screen display module that is presented via the case management application on a computer screen utilizing a modern web browser, such as when summoned using a specific Internet (IP) address. The user screen generally resides in a secure location and requires the operator to authenticate before being permitted access and to designate the patient that is the subject of the medical data records. A patient identifier can be automatically granted for the subject patient by the secure login if the individual is a new, yet unidentified patient, or if the patient is a returning patient, then the system identifies the patient using prior patient identification via, for instance, login credentials (name and password). The patient ID can be retained and then later transferred as individual identifying data when the electronic case abstraction is being compiled. In one embodiment, the server (cognitive engine) can be connected with the user interface via a web widget and REST API, such as noted herein. Both enable the user interface program of a third-party system to be adapted to include the uploader function.

In one or more implementations, once the individual credentials have been approved, a designated graphic can appear on the user's display system that instructs the user to, for instance, locate one or more medical data records that the operator wishes to upload for scanning, or enable the operator to drag and drop medical data records. Any number or type of files can be uploaded, either individually, or collectively. The entire contents of multiple files of a directory or subdirectory can be uploaded in a similar fashion, as can the contents of a CD or a DVD.

In one or more embodiments, files that are uploaded can be transmitted over a secure SSL connection between the user-system and the cognitive engine (e.g., remote server), together with a patient identifier. Once received, the records tagging system can replace any patient identifiers present on the uploaded patient records with the transmitted patient identifier. This can occur differently, depending on the implementation, and depending on the file type. Some file types include a discrete data field with the patient identifier, and some do not. When a file includes a preexisting identifier, it is replaced with the new identifier, and if the file does not include a preexisting identifier, a tag can be added to the filename, ensuring that all files are designated for the same patient case.

In one or more embodiments, tagged cases can then be sorted into different categories based upon file types, preparing the files for future routing to the appropriate file archive storage location. The storage destination can be a vender neutral archive, or VNA. Files are interrogated and sorted by their file extension or suffix first, in one embodiment. Files with standard suffixes for medical image files (DICOM), pathology slides, CCDAs, or text files, can be sorted. Files with suffixes that are indeterminate, such as .PDF files, and certain graphic file extensions such as .TIFF, can be evaluated by the cognitive engine using specified artificial intelligence (AI) rules based on key identifying characteristics to determine how best to categorize the indeterminate file type.

Each file that is uploaded can be scanned for completeness, malware, virus, or inappropriate record type. Executable program file types are not accepted, quarantined and deleted, in one embodiment. Further, an incomplete upload can generate an error condition, and files that are found to be infected can be quarantined with a specific error condition code.

In one or more embodiments, uploaded files can be stored either temporarily, or permanently, depending on the system configuration. Any changes made in the tagging subsystem can be recorded and stored, as well as the type and route on which they are directed in the next processing stage.

In one or more embodiments, a set of rules can be preestablished for the routing of successfully uploaded files. The destination can generally be a VNA that is file type dependent. For example, a medical image file can be routed to a DICOM VNA, and the system can include a set of widget connectors to enable the receiving system to accept the routed file. In one or more embodiments, an audit subsystem can be provided to record the type and date that files have been uploaded, the operator of the uploader, the route that the file has taken, and whether the file has been viewed, scanned for malware, and/or stored.

By way of further example, FIG. 10 depicts a computer system 1000 in communication with external device(s) 1012, which can be used to implement one or more aspects disclosed herein. Computer system 1000 includes one or more processor(s) 1002, for instance central processing unit(s) (CPUs). A processor can include functional components used in the execution of instructions, such as functional components to fetch program instructions from locations such as cache or main memory, decode program instructions, and execute program instructions, access memory for instruction execution, and write results of the executed instructions. A processor 1002 can also include one or more registers to be used by one or more of the functional components. Computer system 1000 also includes a memory 1004, input/output (I/O) devices 1008, and I/O interfaces 1010, which may be coupled to processor(s) 1002 and each other via one or more buses and/or other connections. Bus connections represent one or more of any of several types of bus structures, including a memory bus or memory controller, a peripheral bus, an accelerated graphics port, and a processor or local bus using any of a variety of bus architectures. By way of example, and not limitation, such architectures include the Industry Standard Architecture (ISA), the Micro Channel Architecture (MCA), the Enhanced ISA (EISA), the Video Electronics Standards Association (VESA) local bus, and Peripheral Component Interconnect (PCI).

Memory 1004 can be, or include, main or system memory (e.g. Random Access Memory) used in the execution of program instructions, a storage device(s) such as hard drive(s), flash media, or optical media as examples, and/or cache memory, as examples. Memory 1004 can include, for instance, a cache, such as a shared cache, which may be coupled to local caches (examples include L1 cache, L2 cache, etc.) of processor(s) 1002. Additionally, memory 1004 can be, or include, at least one computer program product having a set (e.g., at least one) of program modules, instructions, code or the like that is/are configured to carry out functions of embodiments described herein when executed by one or more processors.

Memory 1004 can store an operating system 1005 and other computer programs 1006, such as one or more computer programs/applications that execute to perform aspects described herein. Specifically, programs/applications can include computer readable program instructions that can be configured to carry out functions of embodiments of aspects described herein.

Examples of I/O devices 1008 include but are not limited to microphones, speakers, Global Positioning System (GPS) devices, cameras, lights, accelerometers, gyroscopes, magnetometers, sensor devices configured to sense light, proximity, heart rate, body and/or ambient temperature, blood pressure, and/or skin resistance, and activity monitors. An I/O device can be incorporated into the computer system as shown, though in some embodiments an I/O device can be regarded as an external device (812) coupled to the computer system through one or more I/O interfaces 1010.

Computer system 1000 can communicate with one or more external devices 1012 via one or more I/O interfaces 1010. Example external devices include a keyboard, a display, one or more data sensors, and/or any other devices that (for instance) enable a user to interact with computer system 1000. Other example external devices include any device that enables computer system 1000 to communicate with one or more other computing systems or peripheral devices. A network interface/adapter is an example I/O interface that enables computer system 1000 to communicate with one or more networks, such as a local area network (LAN), a general wide area network (WAN), and/or a public network (e.g., the Internet), providing communication with other computing devices or systems, storage devices, or the like. Ethernet-based (such as Wi-Fi) interfaces and Bluetooth® adapters are just examples of the currently available types of network adapters used in computer systems. (BLUETOOTH® is a registered trademark of Bluetooth SIG, Inc., Kirkland, Wash., U.S.A.)

Communication between I/O interfaces 1010 and external devices 1012 can occur across wired and/or wireless communications link(s) 1011, such as Ethernet-based wired or wireless connections. Example wireless connections include cellular, Wi-Fi, Bluetooth®, proximity-based, near-field, or other types of wireless connections. More generally, communications link(s) 1011 can be any appropriate wireless and/or wired communication link(s) for communicating data between systems and/or devices to facilitate one or more aspects disclosed herein.

A particular external device(s) 1012 can include one or more data storage devices, which can store one or more programs, one or more computer readable program instructions, and/or data, etc. Computer system 1000 can include and/or be coupled to and in communication with (e.g., as an external device of the computer system) removable/non-removable, volatile/non-volatile computer system storage media. For example, it can include and/or be coupled to a non-removable, non-volatile magnetic media (typically called a “hard drive”), a magnetic disk drive for reading from and writing to a removable, non-volatile magnetic disk, and/or an optical disk drive for reading from or writing to a removable, non-volatile optical disk, such as a CD-ROM, DVD-ROM or other optical media.

Computer system 1000 can be operational with numerous other general purpose or special purpose computing system environments or configurations. Computer system 1000 can take any of various forms, well-known examples of which include, but are not limited to, personal computer (PC) system(s), server computer system(s), thin client(s), thick client(s), workstation(s), laptop(s), handheld device(s), mobile device(s)/computer(s), such as smartphone(s), tablet(s), and wearable device(s), multiprocessor system(s), microprocessor-based system(s), network appliance(s) (such as edge appliance(s)), virtualization device(s), storage controller(s), set top box(es), programmable consumer electronic(s), network PC(s), minicomputer system(s), mainframe computer system(s), and distributed cloud computing environment(s) that include any of the above systems or devices, and the like.

As will be appreciated by one skilled in the art, control aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system”. Furthermore, control aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable signal medium may be any non-transitory computer readable medium that is not a computer readable storage medium and that can communicate, propagate, or transport a program for use by or in connection with an instruction execution system, apparatus or device.

A computer readable storage medium may be, for example, but not limited to, an electronic, magnetic, optical, electromagnetic, infrared or semiconductor system, apparatus, or device, or any suitable combination of the foregoing. More specific examples (a non-exhaustive list) of the computer readable storage medium include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

In one example, a computer program product includes, for instance, one or more computer readable storage media to store computer readable program code means or logic thereon to provide and facilitate one or more aspects of the present invention.

Program code embodied on a computer readable medium may be transmitted using an appropriate medium, including but not limited to wireless, wireline, optical fiber cable, RF, etc., or any suitable combination of the foregoing.

Computer program code for carrying out control and/or calibration operations for aspects of the present invention may be written in any combination of one or more programming languages, including an object oriented programming language, such as Java, Smalltalk, C++ or the like, and conventional procedural programming languages, such as the “C” programming language, assembler or similar programming languages. The program code may execute entirely on the user's computer, partly on the user's computer, on the user's personal device (e.g., phone, tablet, wearable), as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described herein with reference to block diagrams of methods, apparatus (systems) and computer program products according to embodiments of the invention. It will be understood that the control block of the diagram can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus (e.g., mobile device/phone), or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The block diagram in the figure illustrates the architecture, functionality, and operation of possible implementations of systems, methods and computer program products according to various embodiments of the present invention. In this regard, one or more blocks in the diagram may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that one or more blocks of the diagram can be implemented by special purpose hardware-based systems that perform the specified functions or acts, or combinations of special purpose hardware and computer instructions.

In addition to the above, one or more aspects of the present invention may be provided, offered, deployed, managed, serviced, etc. by a service provider who offers management of customer environments. For instance, the service provider can create, maintain, support, etc. computer code and/or a computer infrastructure that performs one or more aspects of the present invention for one or more customers. In return, the service provider may receive payment from the customer under a subscription and/or fee agreement, as examples. Additionally or alternatively, the service provider may receive payment from the sale of advertising content to one or more third parties.

In one aspect of the present invention, an application may be deployed for performing one or more aspects of the present invention. As one example, the deploying of an application comprises providing computer infrastructure (including, e.g., internet/cloud/TOT resources and/or a mobile device) operable to perform one or more aspects of the present invention.

As a further aspect of the present invention, a computing infrastructure may be deployed comprising integrating computer readable code into a computing system, in which the code in combination with the computing system is capable of performing one or more aspects of the present invention.

As yet a further aspect of the present invention, a process for integrating computing infrastructure comprising integrating computer readable code into a computer system may be provided. The computer system comprises a computer readable medium, in which the computer medium comprises one or more aspects of the present invention. The code in combination with the computer system is capable of performing one or more aspects of the present invention.

A popular type of computing is cloud computing, in which resources can interact and/or be accessed via a communications system, such as a computer network. Resources can be software-rendered simulations and/or emulations of computing devices, storage devices, applications, and/or other computer-related devices and/or services run on one or more computing devices, such as a server. For example, a plurality of servers can communicate and/or share information that can expand and/or contract across servers depending on an amount of processing power, storage space, and/or other computing resources needed to accomplish the requested task.

Cloud computing can be provided as a service over the Internet, such as in the form of “Infrastructure as a Service” (IaaS), “Platform as a Service” (PaaS), and/or “Software as a Service” (SaaS). IaaS can typically provide physical or virtual computing devices and/or accessories on a fee-for-service basis, and onto which clients/users can load and/or install, and management, platforms, applications, and/or data. PaaS can deliver a computing platform and solution stack as a service, such as, for example, a software development platform, application services, such as team collaboration, web service integration, database integration, and/or developer community facilitation. SaaS can deploy software licensing as an application to customers for use as a service on-demand. SaaS software vendors can host the application on their own clouds, or download such applications from clouds to cloud clients, disabling the applications after use, or after an on-demand contract expires.

The provision of such services allows a user access to as much in the way of computing resources as a user needs without purchasing and/or maintaining the infrastructure, such as hardware and/or software, that would be required to provide the services. For example, a user can instead obtain access via subscription, purchase, and/or otherwise securing access. Thus, cloud computing can be a cost-effective way to delivery information technology services.

Although various embodiments are described above, these are only examples. For example, computing environments of other architectures can incorporate and use one or more aspects of the present invention. Additionally, the network of nodes can include additional nodes, and the nodes can be the same or different from those described herein. Also, many types of communications interfaces may be used.

Further, a data processing system suitable for storing and/or executing program code is usable that includes at least one processor coupled directly or indirectly to memory elements through a system bus. The memory elements include, for instance, local memory employed during actual execution of the program code, bulk storage, and cache memory which provide temporary storage of at least some program code in order to reduce the number of times code must be retrieved from bulk storage during execution.

Input/Output or I/O devices (including, but not limited to, mobile device/phone, keyboards, displays, pointing devices, DASD, tape, CDs, DVDs, thumb drives and other memory media, etc.) can be coupled to the system either directly or through intervening I/O controllers. Network adapters may also be coupled to the system to enable the data processing system to become coupled to other data processing systems or remote printers or storage devices through intervening private or public networks. Modems, cable modems, and Ethernet cards are just a few of the available types of network adapters.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has” and “having”), “include” (and any form of include, such as “includes” and “including”), and “contain” (and any form contain, such as “contains” and “containing”) are open-ended linking verbs. As a result, a method or device that “comprises”, “has”, “includes” or “contains” one or more steps or elements possesses those one or more steps or elements, but is not limited to possessing only those one or more steps or elements. Likewise, a step of a method or an element of a device that “comprises”, “has”, “includes” or “contains” one or more features possesses those one or more features, but is not limited to possessing only those one or more features. Furthermore, a device or structure that is configured in a certain way is configured in at least that way, but may also be configured in ways that are not listed.

The corresponding structures, materials, acts, and equivalents of all means or step plus function elements in the claims below, if any, are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to explain the principles of the invention and the practical application, and to enable others of ordinary skill in the art to understand the invention through various embodiments and the various modifications thereto which are dependent on the particular use contemplated.

Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the following claims.

What is claimed is:

1. A computer-implemented method for facilitating medical care, the computer-implemented method comprising:
scanning, by one or more processors, a set of electronic files for electronic medical data records of an individual related to a medical condition, the scanning identifying electronic medical data records of the individual related to the medical condition which include different file types with different unique identifiers, and which are from different electronic medical records systems;
electronically compiling, by the one or more processors, the identified electronic medical data records of the individual related to the medical condition into an electronic case abstraction, including associating a common case identifier with the identified electronic medical data records of the individual related to the medical condition; and
electronically providing, by the one or more processors, for display on a display screen in a consolidated view, the electronic case abstraction to facilitate medical care of the individual.

2. The computer-implemented method of claim 1, wherein the identified electronic medical data records of the individual related to the medical condition compiled for the electronic case abstraction of different file types include two or more of an electronic pathology-type file, an electronic radiology-type file, and an electronic clinical-type file.

3. The computer-implemented method of claim 1, wherein the scanning comprises electronically scanning the set of electronic files using a heuristic algorithm and a database of known medical record patterns, to identify the electronic medical data records of the individual related to the medical condition which include different file types with different unique identifiers, and which are from different electronic medical records systems.

4. The computer-implemented method of claim 1, further comprising obtaining, by the one or more processors, the set of electronic files from the different electronic medical records systems, wherein two or more electronic medical records systems of the different electronic medical records systems are incompatible electronic medical records systems.

5. The computer-implemented method of claim 1, further comprising categorizing by file type and file context the identified electronic medical data records of the individual related to the medical condition, wherein categorizing by file context results in associating two or more identified electronic medical data records of different file type together in the electronic case abstraction.

6. The computer-implemented method of claim 5, wherein the categorizing comprises comparing electronic files of the set of electronic files to known file structures, including known pathology record structures, known radiology record structures, and known clinical record structures to facilitate categorizing the identified electronic medical data records by file type.

7. The computer-implemented method of claim 6, further comprising rules-based forwarding of the identified electronic medical data records to data storage in accordance with respective categorized file types of the identified electronic medical data records.

8. The computer-implemented method of claim 1, wherein the electronically compiling, by the one or more processors, the identified electronic medical data records of the individual related to the medical condition into the electronic case abstraction comprises electronically compiling, by the one or more processors, the electronic case abstraction by extracting metadata from the identified electronic medical data records of the individual related to the medical condition, and processing the metadata to facilitate the electronically compiling of the electronic case abstraction, wherein extracting the metadata includes parsing structured metadata associated with one or more electronic medical data records of the identified electronic medical data records.

9. The computer-implemented method of claim 8, further comprising using a natural language processor to extract unstructured metadata from one or more other electronic medical data records of the identified electronic medical data records to obtain parsed metadata for the other electronic medical data record(s) for use in electronically compiling the electronic case abstraction.

10. The computer-implemented method of claim 1, further comprising:
training one or more machine learning models to make one or more recommendations to facilitate medical care of individuals based on electronic case abstractions; and
electronically providing, by the one or more processors, using the machine learning model(s), a recommendation to facilitate medical care of the individual related to the medical condition.

11. The computer-implemented method of claim 10, wherein the electronically providing comprises electronically providing, by the one or more processors, using the machine learning model(s), a medical referral for the individual related to the medical condition to a specialist.

12. A computer system for facilitating medical care, the computer system comprising:
a memory; and
one or more processors in communication with the memory, wherein the computer system is configured to perform a method comprising:
scanning, by one or more processors, a set of electronic files for electronic medical data records of an individual related to a medical condition, the scanning identifying electronic medical data records of the individual related to the medical condition which include different file types with different unique identifiers, and which are from different electronic medical records systems;
electronically compiling, by the one or more processors, the identified electronic medical data records of the individual related to the medical condition into an electronic case abstraction, including associating a common case identifier with the identified electronic medical data records of the individual related to the medical condition; and
electronically providing, by the one or more processors, for display on a display screen in a consolidated view, the electronic case abstraction to facilitate medical care of the individual.

13. The computer system of claim 12, wherein the identified electronic medical data records of the individual related to the medical condition compiled for the electronic case abstraction of different file types include two or more of an electronic pathology-type file, an electronic radiology-type file, and an electronic clinical-type file.

14. The computer system of claim 12, wherein the scanning comprises electronically scanning the set of electronic files using a heuristic algorithm and a database of known medical record patterns, to identify the electronic medical data records of the individual related to the medical condition which include different file types with different unique identifiers, and which are from different electronic medical records systems.

15. The computer system of claim 12, further comprising categorizing by file type and file context the identified electronic medical data records of the individual related to the medical condition, wherein categorizing by file context results in associating two or more identified electronic medical data records of different file type together in the electronic case abstraction.

16. The computer system of claim 15, wherein the categorizing comprises comparing electronic files of the set of electronic files to known file structures, including known pathology record structures, known radiology record structures, and known clinical record structures to facilitate categorizing the identified electronic medical data records by file type.

17. The computer system of claim 12, wherein the electronically compiling, by the one or more processors, the identified electronic medical data records of the individual related to the medical condition into the electronic case abstraction comprises electronically compiling, by the one or more processors, the electronic case abstraction by extracting metadata from the identified electronic medical data records of the individual related to the medical condition, and processing the metadata to facilitate the electronically compiling of the electronic case abstraction, wherein extracting the metadata includes:
parsing structured metadata associated with one or more electronic medical data records of the identified electronic medical data records; and
using a natural language processor to extract unstructured metadata from one or more other electronic medical data records of the identified electronic medical data records to obtain parsed metadata for the other electronic medical data record(s) for use in electronically compiling the electronic case abstraction.

18. A computer program product for facilitating medical care, the computer program product comprising:
a computer-readable storage medium readable by a processing circuit and storing instructions for execution by the processing circuit for performing a method comprising:
scanning, by one or more processors, a set of electronic files for electronic medical data records of an individual related to a medical condition, the scanning identifying electronic medical data records of the individual related to the medical condition which include different file types with different unique identifiers, and which are from different electronic medical records systems;
electronically compiling, by the one or more processors, the identified electronic medical data records of the individual related to the medical condition into an electronic case abstraction, including associating a common case identifier with the identified electronic medical data records of the individual related to the medical condition; and
electronically providing, by the one or more processors, for display on a display screen in a consolidated view, the electronic case abstraction to facilitate medical care of the individual.

19. The computer program product of claim 18, wherein the identified electronic medical data records of the individual related to the medical condition compiled for the electronic case abstraction of different file types include two or more of an electronic pathology-type file, an electronic radiology-type file, and an electronic clinical-type file.

20. The computer program product of claim 18, wherein the scanning comprises electronically scanning the set of electronic files using a heuristic algorithm and a database of known medical record patterns, to identify the electronic medical data records of the individual related to the medical condition which include different file types with different unique identifiers, and which are from different electronic medical records systems.
